(19) United States
(12) Patent Application Publication
(10) Pub. No.: US 2021/0220231 A1
(43) Pub. Date: Jul. 22, 2021

(54) TWO-LAYERED OIL-BASED COSMETIC

(71) Applicant: Shiseido Company, Ltd., Chuo-ku, Tokyo (JP)

(72) Inventors: Aya CHIKATOMO, Tokyo (JP); Taku TAMARU, Tokyo (JP); Shoko OGAWA, Tokyo (JP); Tomoko IKEDA, Tokyo (JP)

(73) Assignee: Shiseido Company, Ltd., Chuo-ku, Tokyo (JP)

(21) Appl. No.: 17/055,297

(22) PCT Filed: May 16, 2019
(86) PCT No.: PCT/JP2019/019439

§ 371 Date: Nov. 13, 2020

(30) Foreign Application Priority Data
May 22, 2018 (JP) 2018-098327

(51) Int. Cl. A61K 8/03; A61K 8/25; A61K 8/891; A61Q 19/00

(52) U.S. Cl. CPC A61K 8/03; A61K 8/25; A61K 2800/10; A61Q 19/00; A61K 2800/654; A61K 8/891

(57) ABSTRACT

A cosmetic according to the present invention, an objective of which is to provide a two-layered oil-based cosmetic that, despite having a high powder content, is resistant to caking, has excellent powder re-dispersibility and has an excellent texture when applied, is a two-layered oil-based cosmetic containing (a) a non-spherical organopolysiloxane elastomer, (b) silicic anhydride and (c) a powder other than (b) above. Furthermore, the cosmetic of the present invention has a viscosity of 10,000 mPa·s or lower.

### TECHNICAL FIELD

The present invention relates to an oil-based cosmetic having a high powder content. More specifically, the present invention relates to a cosmetic that, despite having a high powder content, prevents caking of the powder and has excellent powder re-dispersibility.

### BACKGROUND ART

In the cosmetic products field, powders are blended for purposes such as coloring the skin, ameliorating skin roughness, and improving the feeling in use when applying the cosmetic to the skin. In particular, oil-based cosmetics are prepared not only as makeup cosmetics, but also as skin-care cosmetics and the like, for reasons such as being able to obtain a smooth feel when applied and the like. However, in oil-based cosmetics, powders are sometimes blended to prepare two-layered or multi-layered oil-based cosmetics in order to suppress shininess and obtain a matte texture when the cosmetics are applied to the skin.

However, if an oil-based cosmetic in which a powder has been blended is placed at rest, there are problems in that the powder ingredients can settle and the settled powder can aggregate and form larger dispersed particles, or the powder can harden and undergo caking, entering a state in which re-dispersion is difficult.

For example, Patent Document 1 describes a multi-layered oil-based cosmetic that suppresses powder aggregation and caking, and that has good powder re-dispersibility due to blending a powder together with silicic anhydride in a liquid oil. However, the amount of the powder that may be blended is indicated as being 0.1% to 10% by mass, and it is unclear whether or not an oil-based cosmetic that suppresses caking and that has good powder re-dispersion is obtained even in cases in which more than 10% by mass of the powder is blended.

An oil-based cosmetic in which more powder can be blended than in conventional cosmetics and having a good texture is sought.

### RELATED ART

### Patent Documents

Patent Document 1: JP 2015-155393 A

### SUMMARY OF THE INVENTION

### Problem to be Solved by the Invention

An objective of the present invention is to provide an oil-based cosmetic that, despite having a high powder content, is resistant to caking and has an excellent texture when applied.

### Means for Solving the Problem

As a result of diligent research towards solving the above-mentioned problem, the present inventors discovered that, by blending a combination of a non-spherical organopolysiloxane elastomer and silicic anhydride with an oil-based cosmetic having a high powder content, the cosmetic has excellent re-dispersibility, is resistant to caking and has excellent spreadability when applied to the skin, despite having a high powder content, thereby completing the present invention.

In other words, the present invention provides a two-layered oil-based cosmetic containing:

(a) a non-spherical organopolysiloxane elastomer;
(b) silicic anhydride; and
(c) a powder other than ingredient (b).

Furthermore, the present invention has the feature wherein the viscosity of the cosmetic overall is 10,000 mPa·s or lower.

### Effects of the Invention

By being configured as mentioned above, the present invention can result in a cosmetic that, despite having a high powder content, has excellent re-dispersibility of the powder and is resistant to caking. Additionally, by combining (a) a non-spherical organopolysiloxane elastomer and (b) silicic anhydride, the cosmetic is made to have a low viscosity at the time of use (when applied to the skin) despite having a high viscosity when at rest, thereby allowing a cosmetic with good spreadability on the skin to be obtained.

### MODES FOR CARRYING OUT THE INVENTION

The two-layered oil-based cosmetic according to the present invention, when left at rest, separates into an upper layer of oil-based ingredients and a lower layer of powder ingredients, thus becoming two-layered. When shaken at the time of use, the two layers mix together, the powder ingredients disperse uniformly in the oil-based ingredients, and the cosmetic exhibits a liquid form.

The two-layered oil-based cosmetic has the feature of containing (a) a non-spherical organopolysiloxane elastomer, (b) silicic anhydride, and (c) a powder other than ingredient (b). Hereinafter, the respective ingredients constituting the cosmetic in the present invention will be explained in detail.

### <(a) Non-Spherical Organopolysiloxane Elastomer>

The (a) non-spherical organopolysiloxane elastomer (hereinafter sometimes referred to simply as “ingredient (a)”) blended into the cosmetic according to the present invention refers to a three-dimensionally crosslinked organopolysiloxane elastomer (silicone elastomer), including those of emulsifying and non-emulsifying type, having organic groups appended to siloxane bonds, and not exhibiting a spherical shape when observed under an optical microscope in a swollen form swollen with an arbitrary solvent. Not exhibiting a spherical shape, in other words, non-spherical shapes, include shapes that are non-uniform.

Emulsifying crosslinked siloxane elastomers, though not particularly limited, include crosslinked polyoxyethylene methylpolysiloxane, alkyl group-containing crosslinked polyoxyethylene methylpolysiloxane, crosslinked polyglycerin-modified silicone, alkyl group-containing crosslinked polyglycerin-modified silicone and the like. As these emulsifying crosslinked siloxane elastomers, those that are commercially available in swollen form, swollen with various types of oils such as silicone oils, mineral oils, triethylhexanoin and squalane, may be used. Specific examples include those indicated below.

Examples of swollen polyoxyethylene methylpolysiloxane crosspolymers include KSG-210 (a mixture of ((PEG-10/15)/dimethicone) crosspolymer and dimethicone, 20% to 30% crosslinked material, manufactured by Shin-Etsu Chemical), 9011 silicone elastomer blend (a mixture of (PEG-12/dimethicone) crosspolymer and cyclomethicone, manufactured by Dow Corning Toray) and the like.

Examples of swollen alkyl group-containing polyoxyethylene methylpolysiloxane crosspolymers include KSG-310 (a mixture of (PEG-15/lauryl dimethicone) crosspolymer and mineral oil, 25% to 35% crosslinked material), KSG-320 (a mixture of (PEG-15/lauryl dimethicone) crosspolymer and isododecane, 20% to 30% crosslinked material), KSG-330 (a mixture of (PEG-15/lauryl dimethicone) crosspolymer and triethylhexanoin, 15% to 25% crosslinked material), KSG-340 (a mixture of (PEG-15/lauryl dimethicone) crosspolymer, (PEG-10/lauryl dimethicone) crosspolymer and squalane, 25% to 35% crosslinked material) (the above all manufactured by Shin-Etsu Chemical) and the like.

Examples of swollen polyglycerin-modified silicone crosspolymers include KSG-710 (a mixture of (dimethicone/polyglyercin-3) crosspolymer and dimethicone, 20% to 30% crosslinked material, manufactured by Shin-Etsu Chemical) and the like.

Examples of swollen alkyl group-containing polyglycerin-modified silicone crosspolymers include KSG-810 (a mixture of (lauryl dimethicone/polyglyercin-3) crosspolymer and mineral oil, 25% to 35% crosslinked material), KSG-820 (a mixture of (lauryl dimethicone/polyglyercin-3) crosspolymer and isododecane, 20% to 30% crosslinked material), KSG-830 (a mixture of (lauryl dimethicone/polyglyercin-3) crosspolymer and triethylhexanoin, 15% to 25% crosslinked material), KSG-840 (a mixture of (lauryl dimethicone/polyglyercin-3) crosspolymer and squalane, 25% to 35% crosslinked material) (the above all manufactured by Shin-Etsu Chemical) and the like.

Non-emulsifying crosslinked siloxane elastomers, though not particularly limited, include methyl polysiloxane crosspolymer, methylphenyl polysiloxane crosspolymer, vinyl dimethicone/lauryl dimethicone crosspolymer, lauryl polydimethylsiloxyethyl dimethicone/bis-vinyl dimethicone crosspolymer, alkyl (C30-45) cetearyl dimethicone crosspolymer, cetearyl dimethicone crosspolymer, (dimethicone/bis-isobutyl PPG-20) crosspolymer and the like. As these non-emulsifying crosslinked siloxane elastomers, those that are commercially available in swollen form, swollen with various types of oils such as silicone oils, mineral oils, triethylhexanoin and squalane, may be used. Specific examples include those indicated below.

Examples of swollen methyl polysiloxane crosspolymers include swollen dimethicone crosspolymers such as 9040 silicone elastomer blend (a mixture of dimethicone crosspolymer and cyclopentasiloxane, 12% crosslinked material), 9041 silicone elastomer blend (a mixture of dimethicone crosspolymer and dimethicone (5 mPa·s), 16% crosslinked material), 9045 silicone elastomer blend (a mixture of dimethicone crosspolymer and cyclopentasiloxane, 12.5% crosslinked material) and EL-8040ID silicone organic blend (a mixture of dimethicone crosspolymer and isododecane, 18% crosslinked material) (the above all manufactured by Dow Corning Toray), swollen dimethicone/vinyl dimethicone crosspolymers such as KSG-15 (a mixture of (dimethicone/vinyl dimethicone) crosspolymer and cyclopentasiloxane, 4% to 10% crosslinked material), KSG-16 (a mixture of (dimethicone/vinyl dimethicone) crosspolymer and dimethicone (6 mPa·s), 20% to 30% crosslinked material), KSG-1610 (a mixture of (dimethicone/vinyl dimethicone) crosspolymer and methyl trimethicone, 15% to 20% crosslinked material) (the above all manufactured by Shin-Etsu Chemical) and CXG-1101 (a mixture of (dimethicone/vinyl dimethicone) crosspolymer and cyclopentasiloxane, 12% crosslinked material, with a viscosity of 550,000 mPa·s, manufactured by NuSil Technology), and the like.

Examples of swollen methylphenyl polysiloxane crosspolymers include KSG-18A (a mixture of (dimethicone/phenylvinyl dimethicone) crosspolymer and diphenylsiloxyphenyl trimethicone, 10% to 20% crosslinked material) (manufactured by Shin-Etsu Chemical) and the like.

Examples of swollen vinyl dimethicone/lauryl dimethicone crosspolymers include KSG-41A (a mixture of (vinyl dimethicone/lauryl dimethicone) crosspolymer and mineral oil, 20% to 30% crosslinked material), KSG-42A (a mixture of (vinyl dimethicone/lauryl dimethicone) crosspolymer and isododecane, 15% to 25% crosslinked material), KSG-43 (a mixture of (vinyl dimethicone/lauryl dimethicone) crosspolymer and triethylhexanoin, 25% to 35% crosslinked material), KSG-44 (a mixture of (vinyl dimethicone/lauryl dimethicone) crosspolymer and squalane, 25% to 35% crosslinked material) (the above all manufactured by Shin-Etsu Chemical) and the like.

Examples of swollen lauryl polydimethylsiloxyethyl dimethicone/bis-vinyl dimethicone crosspolymers include KSG-042Z (a mixture of (lauryl polydimethylsiloxyethyl dimethicone/bis-vinyl dimethicone) crosspolymer and isododecane, approximately 20% crosslinked material), KSG-045Z (a mixture of (lauryl polydimethylsiloxyethyl dimethicone/bis-vinyl dimethicone) crosspolymer and cyclopentasiloxane, approximately 20% crosslinked material) (the above all manufactured by Shin-Etsu Chemical) and the like.

Examples of swollen alkyl (C30-45) cetearyl dimethicone crosspolymers include VELVESIL 125 (a mixture of alkyl (C30-45) cetearyl dimethicone crosspolymer and cyclopentasiloxane, approximately 12.5% crosslinked material), VELVESIL 034 (a mixture of alkyl (C30-45) cetearyl dimethicone crosspolymer and caprylyl methicone, approximately 16% crosslinked material) (the above all manufactured by Momentive Performance Materials) and the like.

Examples of swollen cetearyl dimethicone crossplymers include VELVESIL DM (a mixture of cetearyl dimethicone crosspolymer and dimethicone (5 mPa·s), approximately 17% crosslinked material, manufactured by Momentive Performance Materials) and the like.

Examples of swollen (dimethicone/bis-isobutyl PPG-20) crosspolymers include EL-8050ID Silicone Organic Elastomer Blend (a mixture of (dimethicone/bis-isobutyl PPG-20) crosspolymer and isododecane, 15% crosslinked material, manufactured by Dow Corning Toray) and the like.

As mentioned above, the non-spherical organopolysiloxane elastomers according to the present invention are commercially available in swollen form, swollen with various types of oils such as silicone oils, mineral oils, triethylhexanoin and squalane. In the cosmetic of the present invention, they are preferably blended in a swollen form swollen with a silicone oil. Furthermore, the non-spherical organopolysiloxane elastomer of the present invention, when dispersed in cyclopentasiloxane so as to be 3% in terms of solid content, preferably has a viscosity (30° C.) of 5 to 10,000 mPa·s, more preferably 200 to 5,000 mPa·s, and even more preferably 1,000 to 5,000 mPa·s.

In particular, the non-spherical organopolysiloxane elastomer of the present invention is preferably a swollen methyl polysiloxane crosspolymer, more preferably CXG-1101 (a mixture of (dimethicone/vinyl dimethicone) crosspolymer and cyclopentasiloxane, 12% crosslinked material, with a viscosity of 550,000 mPa·s, manufactured by NuSil Technology), KSG-16 (a mixture of (dimethicone/vinyl dimethicone) crosspolymer and dimethicone (6 mPa·s), 20% to 30% crosslinked material, manufactured by Shin-Etsu Chemical) or KSG-15 (a mixture of (dimethicone/vinyl dimethicone) crosspolymer and cyclopentasiloxane, 4% to 10% crosslinked material, manufactured by Shin-Etsu Chemical).

The ingredient (a) in the present invention may be one of the aforementioned elastomers used alone, or may be a combination of two or more.

The blended amount of ingredient (a) should be 1% to 20% by mass, preferably 3% to 18% by mass, and more preferably 5% to 12% by mass relative to the total mass of the cosmetic. If the blended amount of ingredient (a) is less than 1% by mass, then the re-dispersibility of the powder when shaken becomes worse, and if more than 20% by mass is blended, then the spreadability of the cosmetic when applied to the skin becomes poor.

### <(b) Silicic Anhydride>

The (b) silicic anhydride (hereinafter sometimes referred to simply as “ingredient (b)”) blended into the cosmetic according to the present invention is not particularly limited, and only needs to be a silicic anhydride (silica) that can normally be used in cosmetics. Additionally, the shape of the silicic anhydride in the present invention is not particularly limited and may be any shape, such as spherical, flake-shaped or rod-shaped. Although the size of the silicic anhydride used in the present invention is not particularly limited, the average primary particle size should preferably be 1 to 100 nm, more preferably 5 to 50 nm. In this case, the “average primary particle size” in the present invention refers to the size of the primary particles measured by a method that is generally used in the cosmetics field. Specifically, it is a value determined as the arithmetic mean of the long axes and the short axes of the particles as determined from transmission electron microscope photographs, laser scattering/diffraction or the like.

Silicic anhydrides that can be used in cosmetics include surface-untreated hydrophilic silicic anhydrides, hydrophobic silicic anhydrides that have been surface-treated with hydrophobic substances and the like, either type being usable as ingredient (b) in the present invention. In the cosmetic of the present invention, a hydrophobic silicic anhydride that has been surface-treated with a hydrophobic substance is preferably used.

In the hydrophobic silicic anhydride in the present invention, silanol groups (hydroxy groups) on the surfaces of the silicic anhydride are dimethyl dichlorosilane-treated, octylsilane-treated, hexamethyl disilazane-treated, dimethyl silicone oil-treated or methacryloxysilane-treated. In particular, those that are dimethyl dichlorosilane-treated, octylsilane-treated, hexamethyl disilazane-treated or dimethyl silicone oil-treated are preferred for having excellent cosmetic stability improvement effects.

Examples of commercially available hydrophobic silicic anhydrides include AEROSIL R972, R972V, R972CF, R974, R976 and R976S (manufactured by Nippon Aerosil), which are dimethyl dichlorosilane-treated products, AEROSIL R805 (manufactured by Nippon Aerosil), which is an octylsilane-treated product, AEROSIL R812, R812S and RX200 (manufactured by Nippon Aerosil), which are hexamethyl disilazane-treated products, AEROSIL R202 and RY200 (manufactured by Nippon Aerosil), which are dimethyl silicone oil-treated products, and the like. In the present invention, one or more of the above may be blended and used.

The blended amount of ingredient (b) should be 0.005% to 2.0% by mass, preferably 0.008% to 1.2% by mass, and more preferably 0.008% to 1.0% by mass relative to the total mass of the cosmetic. If the blended amount of ingredient (b) is less than 0.005% by mass, then the re-dispersibility of the powder when shaken becomes worse, and if more than 2.0% by mass is blended, then a tendency to adversely affect the texture, such as a feeling of squeakiness on the skin or poor spreadability when applied, is observed.

### <(c) Powder>

The (c) powder (hereinafter sometimes referred to simply as “ingredient (c)”) blended into the cosmetic according to the present invention is not particularly limited, and refers to a powder ingredient that can normally be blended into cosmetics, excluding the aforementioned ingredient (b). The shape of the powder (c) is not particularly limited and may be any shape, such as spherical, flake-shaped or rod-shaped. Examples include inorganic powders, organic powders and the like.

Examples of inorganic powders include inorganic powders such as talc, kaolin, mica and zeolite; inorganic white pigments such as titanium dioxide and zinc oxide; inorganic red iron oxides such as iron oxide (red ocher); inorganic yellow pigments such as yellow iron oxide and yellow ocher; black pigments such as black iron oxide and carbon black; inorganic green pigments such as chromium oxide, chromium hydroxide and cobalt titanate; inorganic blue pigments such as ultramarine blue and Prussian blue; pearlescent pigments such as titanium oxide-coated mica, colored titanium oxide-coated mica, bismuth oxychloride and argentine; metal powder pigments such as aluminum powder and copper powder; and the like.

Examples of organic powders include organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401 and Blue No. 404; organic pigments such as zirconium, barium or aluminum lakes, such as Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3 and Blue No. 1; resin powders such as polyamide resin (nylon powder) polyethylene powders, polymethyl methacrylate powders, polyurethane powders, polystyrene powders, polyalkyl acrylate powders, styrene and acrylic acid copolymer resin powders, silicone powders and crosslinked silicone powders; and the like. Examples of commercially available resin powders include a polymethyl silsesquioxane powder (product name “Tospearl 2000B”, manufactured by Momentive Performance Materials Japan), a silicone composite powder (product name “KSP100”, manufactured by Shin-Etsu Chemical), a polymethyl methacrylate (product name “Matsumoto Microsphere M-330”, manufactured by Matsumoto Yushi) and the like.

The blended amount of ingredient (c) in the cosmetic of the present invention should be 10% to 50% by mass, preferably 15% to 45% by mass, relative to the total mass of the cosmetic. If the blended amount of ingredient (c) is less than 10% by mass, then there is a strong sense of stickiness, and if more than 50% by mass is blended, then the re-dispersibility becomes worse, and there is a concern of decreases in the texture, such as poor spreadability when applied.

By blending the above-mentioned ingredients (a) to (c), the two-layered oil-based cosmetic according to the present invention provides a cosmetic that has excellent re-dispersibility despite containing more powder than in conventional cosmetics. Additionally, by combining the aforementioned ingredient (a) and ingredient (b), a cosmetic having high viscosity when placed at rest while having low viscosity when applied to the skin can be obtained, thereby allowing an oil-based cosmetic having good spreadability on the skin and having excellent powder dispersibility to be obtained.

The two-layered oil-based cosmetic according to the present invention can be prepared in various formats, such as makeup cosmetics and skin-care cosmetics. In particular, the two-layered oil-based cosmetic of the present invention is particularly novel in that it has a high powder content, and thus can be utilized as a texture-adjusting cosmetic, a few drops of which can be mixed into an already prepared liquid cosmetic at the time of use to change the texture to a preferred texture at the time of application to the skin. For example, the greater the amount of the two-layered oil-based cosmetic of the present invention that is mixed into an existing liquid makeup cosmetic, the more it changes to a matte texture. The liquid cosmetic in this case is not particularly limited, but the two-layered oil-based cosmetic of the present invention can be favorably mixed if the liquid cosmetic is a water-in-oil emulsified cosmetic or an oil-based cosmetic.

Although the favorable viscosity of the cosmetic of the present invention will differ in accordance with the manner of use, from the viewpoint of spreadability on the skin and mixability with other liquid cosmetics, it has the feature wherein the viscosity measured after the entire cosmetic has been shaken approximately twenty times until uniform, under 30° C. conditions using a BL-type viscometer, is 10,000 mPa·s or lower.

Additionally, in the oil-based cosmetic of the present invention, various types of ingredients that are normally used in cosmetics may be blended, as appropriate, within a range not inhibiting the effects of the present invention. Examples include oils, ultraviolet protectants, anionic surfactants, cationic surfactants, amphoteric surfactants, non-ionic surfactants, humectants, water-soluble polymers, filming agents, metal ion sequestrants, lower alcohols, polyhydric alcohols, sugars, amino acids, organic amines, polymer emulsions, pH adjusters, skin nutrients, vitamins, antioxidants, antioxidant aids, microbicidal preservatives, other medicinal agents, colorants, fragrances, water and the like.

The oil blended into the cosmetic of the present invention is not particularly limited and may be an oil that is normally used in cosmetics. Specific examples include liquid oils/fats such as avocado oils, camellia oil, macadamia nut oil, mink oil, olive oil, castor oil, jojoba oil, triglycerin and glycerin trioctanoate; solid oils/fats such as coconut oil, hardened coconut oil, palm oil, beef tallow, sheep tallow, Japan tallow and hardened castor oil; hydrocarbon oils such as liquid paraffin, squalane, paraffin, ceresin, vaseline, squalene and microcrystalline wax; higher fatty acids such as lauric acid, myristic acid, palmitic acid, stearic acid, behenic acid, oleic acid, 12-hydroxystearic acid, isostearic acid, linolic acid and linoleic acid; higher alcohols such as lauryl alcohol, cetyl alcohol, stearyl alcohol, behenyl alcohol, oleyl alcohol, monostearyl glycerol ether, monopalmityl glycerol ether, cholesterol, phytosterol and isostearyl alcohol; ester oils such as isopropyl myristate, cetyl octanoate, octyldodecyl myristate, butyl stearate, decyl oleate, ethylene glycol dioctanoate, diisostearyl malate, trimethylol propane trioctanoate, trimethylol propane triisostearate, pentaerythritol tetraoctanoate, glyceryl trioctanoate, glyceryl triisostareate, ethyl acetate, butyl acetate and amyl acetate; and silicone oils such as dimethyl polysiloxane, methylphenyl polysiloxane, methylhydrogen polysiloxane, cyclopentasiloxane, octamethyl cyclotetrasiloxane, silicone resins forming three-dimensional network structures, silicone rubber and various modified polysiloxanes (such as amino-modified polysiloxane, polyether-modified polysiloxane, alkyl-modified polysiloxane and fluorine-modified polysiloxane). In particular, silicone oils are preferably used in the cosmetic of the present invention. As the oil in the present invention, one type selected from the aforementioned oils may be used, or a combination of two or more types may be used.

The cosmetic of the present invention may be produced by a conventional method used for oil-based cosmetics in which powders are blended.

### EXAMPLES

Although the present invention will be explained in further detail by providing specific examples below, the present invention is not limited to the examples below. Additionally, the blended amounts in the examples, etc. below are expressed in percentage by mass where not indicated otherwise.

The two-layered oil-based cosmetics having the compositions indicated in Table 1 below were prepared by conventional methods, and (1) measured for viscosity, (2) evaluated for powder re-dispersibility and (3) evaluated for spreadability on the skin. The evaluations were performed as indicated below.

### (1) Viscosity Measurement

After the prepared samples were allowed to rest for one day at 30° C., they were shaken approximately twenty times until entirely uniform, and measured for viscosity (mPa·s) by a BL-type viscometer (rotor selected by discretion in accordance with viscosity width, twelve rotations, one minute). The results are shown in the table.

### (2) Evaluation of Re-Dispersibility

After the prepared samples were allowed to rest for one month at room temperature, the states of re-dispersibility of the powder ingredients when the containers were shaken up and down ten times were observed by eye, and evaluated on the basis of the criteria indicated below. The results are shown in the table.

### <Evaluation Criteria>

A: Very good

### B: Good

C: Slightly poor

### D: Poor

### (3) Spreadability on Skin

Actual usage tests were performed by ten expert panelists. The spreadaility when each cosmetic was applied to the skin was evaluated on the basis of the criteria indicated below. The results are shown in the table.

### <Evaluation Criteria>

A: Seven or more of the panelists replied that the spreadability was good.
B: Four to six of the panelists replied that the spreadability was good.
C: Three or fewer of the panelists replied that the spreadability was good.

TABLE 1

| | | | | | | Comp | Comp | Comp | Comp | Comp | Comp |
|---|---|---|---|---|---|---|---|---|---|---|---|
| Ingredient | Ex 1 | Ex 2 | Ex 3 | Ex 4 | Ex 5 | Ex 1 | Ex 2 | Ex 3 | Ex 4 | Ex 5 | Ex 6 |
| Ethyl alcohol | 5.0 | 5.0 | 5.0 | 5.0 | 5.0 | 5.0 | 5.0 | 5.0 | 5.0 | 5.0 | 5.0 |
| Non-spherical | 8.0 | 8.0 | 8.0 | 1.0 | 20 | 8.0 | — | 8.0 | 8.0 | 0.5 | 30 |
| organopolysiloxane | | | | | | | | | | | |
| elastomer *¹ | | | | | | | | | | | |
| Hydrophobically | 0.01 | 0.05 | 1.0 | 0.01 | 0.01 | — | 0.01 | 0.001 | 3.0 | 0.01 | 0.01 |
| treated fumed silicic | | | | | | | | | | | |
| anhydride | | | | | | | | | | | |
| Silicone resin powder | 24.0 | 24.0 | 24.0 | 24.0 | 24.0 | 24.0 | 24.0 | 24.0 | 24.0 | 24.0 | 24.0 |
| Cyclopentasiloxane | 62.99 | 62.95 | 62.0 | 69.99 | 50.99 | 63.0 | 70.99 | 62.999 | 60.0 | 70.49 | 40.99 |
| Viscosity (mPa · s) | 885 | 1174 | 1624 | 63.1 | 7210 | 190 | 35.4 | 345 | 2730 | 42.2 | 32090 |
| Re-dispersibility when | A | B | B | B | B | C | D | C | B | C | B |
| shaken | | | | | | | | | | | |
| Spreadability | A | A | B | A | B | A | A | A | C | A | C |

*¹ CXG-1101

In the oil-based cosmetics with high powder content, the powder re-dispersibility was worse in the case in which ingredient (b) of the present invention was not blended (Comparative Example 1), in the case in which ingredient (a) was not blended (Comparative Example 2), in the case in which the content of ingredient (b) was less than 0.005% by mass (Comparative Example 3) and in the case in which the content of ingredient (a) was less than 1% by mass (Comparative Example 5). Additionally, the spreadability on the skin tended to be worse in the case in which the blended amount of ingredient (b) exceeded 2% by mass (Comparative Example 4) and in the case in which the blended amount of ingredient (a) exceeded 20% by mass (Comparative Example 6). In contrast therewith, the cosmetics of Examples 1 to 5 all had excellent powder re-dispersibility and good spreadability on the skin.

Next, the viscosities of the cosmetic of Example 1 and the cosmetic of Comparative Example 1 were measured after different numbers of rotations and compared. The results are shown in Table 2 below. The viscosity under low rotation conditions (approximately 10 rotations or less) represents the viscosity when the cosmetic is at rest, and the viscosity under high rotation conditions (approximately 80 rotations or more) represents the viscosity when spread on the skin or mixed (when used).

TABLE 2
Comparative Example 1

| No. of Rotations | 3 | 12 | 100 |
|---|---|---|---|
| Viscosity | 640 | 240 | 87.8 |

Example 1

| No. of Rotations | 1.5 | 12 | 100 |
|---|---|---|---|
| Viscosity | 4630 | 1047 | 274.7 |

As shown in Table 2, the cosmetic of Comparative Example 1 had low viscosity under both low rotation conditions and high rotation conditions. This indicates that the cosmetic of Comparative Example 1 has low viscosity both when at rest and when used. Thus, the cosmetic of Comparative Example 1 has good spreadability of the cosmetic at the time of use, but can be expected to be susceptible to settling and caking of the powder when at rest. In contrast therewith, the cosmetic of Example 1 had high viscosity with a low number of rotations, but had low viscosity with a high number of rotations. This indicates that the viscosity was high when at rest and the viscosity was low when used. When at rest, the powder does not tend to settle and it can be easily re-dispersed, and the spreadability at the time of use can be considered to be good.

Hereinafter, other formulation examples of the two-layered oil-based cosmetic according to the present invention will be presented. All of the formulations had excellent powder re-dispersibility and good spreadability on the skin.

### (Formulation Example 1) Texture-Adjusting Oil-Based Cosmetic

| Blended ingredients | % by mass |
|---|---|
| Ethyl alcohol | 5.00% |
| Non-spherical organopolysiloxane elastomer *¹ | 8.00% |
| Silicone resin powder | 24.00% |
| Fumed silicic anhydride | 0.01% |
| Cyclopentasiloxane | 62.99% |

*¹ CXG-1101

### (Formulation Example 21 Texture-Adjusting Oil-Based Cosmetic

| Blended ingredients | % by mass |
|---|---|
| Ethyl alcohol | 5.00% |
| Non-spherical organopolysiloxane elastomer *¹ | 10.00% |
| Silicone resin powder | 10.00% |
| Hydrophobically treated fumed silicic anhydride | 0.01% |
| Cyclopentasiloxane | 74.99% |

*¹ CXG-1101

### (Formulation Example 3) Texture-Adjusting Oil-Based Cosmetic

| Blended ingredients | % by mass |
|---|---|
| Ethyl alcohol | 5.00% |
| Non-spherical organopolysiloxane elastomer *¹ | 7.00% |
| Silicone resin powder | 30.00% |
| Hydrophobically treated fumed silicic anhydride | 0.01% |
| Cyclopentasiloxane | 57.99% |

*¹ CXG-1101

### (Formulation Example 41 Liquid Foundation Cosmetic

| Blended ingredients | % by mass |
|---|---|
| Ethyl alcohol | 5.00% |
| Non-spherical organopolysiloxane elastomer *¹ | 8.00% |
| Silicone resin powder | 22.60% |
| Titanium oxide | 1.00% |
| Yellow iron oxide | 0.20% |
| Black iron oxide | 0.10% |
| Red iron oxide | 0.10% |
| Hydrophobically treated fumed silicic anhydride | 0.01% |
| Cyclopentasiloxane | 62.99% |

*¹ CXG-1101

### (Formulation Example 5) Texture-Adjusting Oil-Based Cosmetic

| Blended ingredients | % by mass |
|---|---|
| Ethyl alcohol | 5.00% |
| Non-spherical organopolysiloxane elastomer *² | 17.00% |
| Silicone resin powder | 24.00% |
| Hydrophobically treated fumed silicic anhydride | 0.01% |
| Cyclopentasiloxane | 53.99% |

*² KSG-15

### (Formulation Example 6) Lip Cosmetic

| Blended ingredients | % by mass |
|---|---|
| Non-spherical organopolysiloxane elastomer *¹ | 8.00% |
| Pearlescent agent | 1.00% |
| Silicone resin powder | 12.00% |
| PMMA powder | 12.00% |
| Hydrophobically treated fumed silicic anhydride | 0.01% |
| Methylphenyl polysiloxane (10 cs) | 66.99% |

*¹ CXG-1101

### (Formulation Example 7) Body Oil

| Blended ingredients | % by mass |
|---|---|
| Ethyl alcohol | 5.00% |
| Non-spherical organopolysiloxane elastomer *¹ | 8.00% |
| Silicone resin powder | 10.00% |
| Mica | 5.00% |
| Pearlescent agent | 0.50% |
| Hydrophobically treated fumed silicic anhydride | 0.01% |
| Cyclopentasiloxane | 71.49% |

*¹ CXG-1101

1. A two-layered oil-based cosmetic containing:
(a) a non-spherical organopolysiloxane elastomer;
(b) silicic anhydride; and
(c) a powder other than ingredient (b).

2. The cosmetic according to claim 1, having a viscosity of 10,000 mPa·s or lower.

3. The cosmetic according to claim 1, wherein the ingredient
(b) is a fine-particle silicic anhydride.

4. The cosmetic according to claim 1,
wherein the ingredient (b) is a hydrophobic fine-particle silicic anhydride.

5. The cosmetic according to claim 1,
containing 10% to 50% by mass of the ingredient (c).

6. The cosmetic according to claim 1,
containing 1% to 20% by mass of the ingredient (a).

7. The cosmetic according to claim 1,
containing 0.005% to 2.0% by mass of the ingredient (b).
